(19) United States
(12) Patent Application Publication
(10) Pub. No.: US 2014/0050321 A1
(43) Pub. Date: Feb. 20, 2014

(54) ULTRASONIC TRANSMISSION OF SIGNALS

(71) Applicants: David E. ALBERT, Oklahoma City, OK (US); James J. LEWIS, Spokane, WA (US); Kim Norman BARNETT, Mt. Tamborine (AU); Bruce Richard SATCHWELL, Carrara (AU)

(72) Inventors: David E. ALBERT, Oklahoma City, OK (US); James J. LEWIS, Spokane, WA (US); Kim Norman BARNETT, Mt. Tamborine (AU); Bruce Richard SATCHWELL, Carrara (AU)

(21) Appl. No.: 13/969,446

(22) Filed: Aug. 16, 2013

Related U.S. Application Data
(60) Provisional application No. 61/684,096, filed on Aug. 16, 2012.
(60) Provisional application No. 61/725,422, filed on Nov. 12, 2012.

(51) Int. Cl. H04W 12/04; A61B 5/0205; H04B 11/00

(52) U.S. Cl. CPC H04W 12/04; H04B 11/00; A61B 5/0205
USPC 380/270; 367/137; 600/324

(57) ABSTRACT

Described herein are devices and systems that transmit data from a first device using an ultrasonic digital modem to a second device that receives the ultrasonic signal and can interpret the ultrasonic signal. The second device may be a telecommunications device such as a smartphone running an ultrasonic digital modem receiver application. In particular, devices, systems and methods for encoding and transmitting an ultrasonic signal that includes both digital (e.g., FSK) and analog signal components. Such hybrid ultrasonic signals may efficiently and reliably transmit information, and particularly biological information. Also described herein are devices, systems and methods for securely transmitting ultrasonic signals using encryption keys that may be read by the receiving device using a separate (e.g., non-ultrasound modality) from the transmitting device.

### CROSS REFERENCE TO RELATED APPLICATIONS

This patent application claims priority to U.S. Provisional Patent Application No. 61/684,096, titled “ULTRASONIC TRANSMISSION OF SIGNALS,” filed on Aug. 16, 2012 and U.S. Provisional Patent Application No. 61/725,422, titled “ULTRASONIC TRANSMISSION OF SIGNALS FROM A BIOMETRIC DATA-SENSING WRISTLET, filed Nov. 12, 2012, each of which is herein incorporated by reference in their entirety.

This material may be related to U.S. patent applications: 12/796,188, now U.S. Pat. No. 8,509,882, titled “HEART MONITORING SYSTEM USABLE WITH A SMART PHONE OR COMPUTER,” filed Jun. 8, 2010 and U.S. patent application No. 13/108,738, titled “WIRELESS, ULTRASONIC PERSONAL HEALTH MONITORING SYSTEM,” filed May 16, 2011, Publication No. US-2011-0301439-A1, each of which is herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This patent application discloses inventive concept(s) related generally to systems, methods and devices, including hardware, firmware and software (and any other non-transitory computer-readable storage medium storing a set of instructions capable of being executed by a computing device including a smartphone), for securely and efficiently ultrasonically communicating information with computing devices and in particular mobile communications devices such as smartphones, tablets and computers. The source of the information may be a user device, including individual monitoring or medical devices.

### BACKGROUND

Consumer products including monitoring devices may record information which may be ultrasonically transmitted to one or more receiving devices located nearby. Ultrasonic transmission shares many similarities with electrical transmission, but there are also substantial differences. In particular, the transmission of ultrasonic data has, to date, been somewhat limited in the informational content. For example, digital encoding of information by ultrasound has been somewhat limited in the amount and content of the information transmitted. There is not yet any standard for transmission or encoding of ultrasonic transmission. Further, such ultrasonic signals are not routinely encrypted.

It would be advantageous to provide systems, devices and methods for encoding or arranging information sent by ultrasonic transmission. In particular, it would be advantageous to encode information in a manner that recognizes both the limits and benefits of ultrasonic (as opposed to electromagnetic) transmission and is specifically adapted to operate with such signals. In addition, it would be helpful to provide methods, devices and systems for securely transmitting (e.g., encrypting and/or decrypting) ultrasonic transmissions. For example, it would be helpful to dynamically pair a device that ultrasonically transmits information with one or more receiving device.

Virtually any device having a tone generator (e.g., a piezoelectric speaker) and a processor/controller (e.g., microcontroller) that can control output from the tone generator may be configured (or reconfigured) as an ultrasonic transmission device. For example, consumer devices (e.g., medical devices for personal use, such as thermometers, glucose monitors, blood pressure cuffs, pulse oximeters, heart rate monitors, activity monitors, pedometers, etc.) are one example of a technology that would benefit from a simple, reliable and cost effective ways to transmit data ultrasonically to a telecommunications device. For example, many medical devices include a digital display to present output. This digital information is not usually transmitted beyond the device. However, in many instances it may be beneficial to transmit the digital medical health information to one or more locations so that the medical information may be accessed and/or manipulated by others. For example, it may be useful for a patient to record and provide access to detected health information (e.g., blood pressure, blood sugar, temperature, telemetry, etc.) to medical professionals. Access may be provided by uploading the medical information to a server and/or website; the website may be used to store, provide remote access to the user and/or qualified medical professionals, or analyze the health information.

Described herein are methods, devices, and systems for using (or adapting for use) one or more widely available telecommunications devices, such as smart phones, tablet computers, portable computers or desktop computers, to receive and send information (including but not limited to digital health information) that has been encoded by an application device into an ultrasonic signal that can be heard by the telecommunications device and then stored, transmitted and/or analyzed by the telecommunications device. In particular, described herein are methods, devices and systems for encoding this information so that it may be interpreted only by a telecommunications device that has been provided a key. The system, devices and methods (including executable logic) may include techniques for readily providing the key using a different modality (e.g., optical) than the ultrasonic transmission.

U.S. patent application No. 12/796,188, now U.S. Pat. No. 8,301,232, titled “HEART MONITORING SYSTEM USABLE WITH A SMART PHONE OR COMPUTER,” filed Jun. 8, 2010 and U.S. patent application No. 13/108,738, titled “WIRELESS, ULTRASONIC PERSONAL HEALTH MONITORING SYSTEM,” filed May 16, 2011, Publication No. US-2011-0301439-A1, describe ECG monitors that convert ECG data into ultrasound signals that can be received by a telecommunications device such as a smartphone and then stored, analyzed, and/or displayed. The instant application extends and adapts this teaching and may be used with any of the systems, methods and devices described herein.

### SUMMARY OF THE DISCLOSURE

In general, described herein are devices, systems and methods for ultrasonically transmitting digital data from (and in some cases to) a device having a microprocessor and a transducer capable of delivering ultrasonic frequencies (i.e., piezo speaker). The digitally transmitted data may be received by a receiving device having a microphone, such as a telecommunications device (e.g., a personal telecommunications device, phone such as an iphone, DROID, or other smartphone, iPad or other personal computers, PDAs, or the like). The digital information transmitted may be encoded and/or encrypted as described in greater detail below. In addition, the information may be compressed (data compressed) before encryption.

Also described herein are ultrasonic digital modems and digital modem protocols and logic for securely transmitting digital information ultrasonically to a receiver, which may be configured as a telecommunications device.

Also described herein are microcontrollers configured as ultrasonic modems. In some variations the microcontrollers include logic (e.g., hardware, software, firmware, or some combination thereof) that permits the device to drive ultrasonic transmission of data from a speaker (e.g., piezoelectric speaker element). Methods of configuring or adapting a microcontroller to operate as an ultrasonic modem are also described. For example, in some variations a microcontroller may be programmed to operate as an ultrasonic modem. The ultrasonic modem may be configured to format the information to be transferred as a hybrid digital and analog format. In some variations the ultrasonic modem may be an ultrasonic modem component that encrypts the information using an encryption key.

Also described herein are receivers configured to receive ultrasonic digital data acoustically transmitted by an ultrasonic digital modem. In general, a telecommunications device (e.g., smartphone) may be configured to act as a receiver to receive ultrasonic digital data. Thus, a telecommunications device may include hardware, software, and/or firmware configured to receive, decode, interpret, display, analyze, store and/or transmit data sent by ultrasonic transmission from a digital ultrasonic modem. In some variations logic (e.g., client software and/or firmware, applications, etc.) may be executed on the telecommunications device so that it may act as a receiver for the digital ultrasound data. Thus, described herein is executable logic for receiving and interpreting (e.g., decoding) data transmitted by digital ultrasonic modem, and devices including executable logic for receiving and interpreting (e.g., decoding) data transmitted by digital ultrasonic modem executable logic. For example, described herein are non-transitory computer-readable storage mediums storing instructions capable of being executed by a computing device, and in particular a smartphone, that when executed by the computing device (e.g., smartphone) causes the smartphone to receive and/or send and interpret (e.g., decoding/encode) data transmitted by digital ultrasonic modem.

Also described herein are systems including a microcontroller configured as a digital ultrasound modem and a telecommunications device with ultrasonic model receiver logic.

Further described herein are specific devices and system configured to include digital ultrasonic modems. Any of these devices may include a source of the digital information (e.g., device such as a medical device (e.g., thermometer, pulse oximiter, etc.), a sound transducer (e.g., a speaker capable of emitting ultrasound signals) and a controller (e.g., microcontroller) configured to encode digital information from the source of digital information as an ultrasound signal to be transmitted by the sound transducer. In some variations the sound transduce is configured to emit both audible (e.g., lower than ultrasound) sounds (to buzz, beep and the like within normal human hearing range) as well as emitting in the ultrasound frequency (e.g., greater than 17 kHz, greater than 18 kHz, greater than 19 kHz, between about 17 kHz and about 40 kHz, between about 17 kHz and about 30 kHz, etc.).

In one example described herein a medical device (e.g., a Texas Instrument's AFE4110 digital thermometer) has been retrofitted and modified as described to encode and transmit the temperature data ultrasonically to a telecommunications device (e.g., a smartphone) located some distance from the thermometer. The microcontroller of the device (an MSP430 type controller from Texas Instruments) has been configured to include an ultrasonic modem for transmission of ultrasonic digital data by encoding (via the microprocessor) the data signal for transmission on a connected piezoelectric speaker. The speaker may be the same speaker that is preset in the device (e.g., thermometer) already used for audibly (e.g., with the normal audible range for humans) notifying the user that the temperature is stable. Thus, a device (such as a thermometer) may be retrofitted to include the digital ultrasound modem at very low cost by including control logic in the microcontroller to process data from the thermometer and transmit the encoded signal on the piezoelectric speaker in the ultrasonic frequency range (e.g., >17 KHz). A device transmitting ultrasonically may encode the ultrasonically transmitted information, and may include a security key printed on the outside of the device (e.g., as a bar code, QR code, etc.) that may be read by the receiving telecommunications device (e.g., smartphone) and used to pair the devices and/or decode the transmitted information.

For example, described herein are medical sensing apparatuses (devices and systems) that use ultrasound to digitally transmit biological parameters received by the medical sensing device to one or more telecommunications devices (e.g., a smartphone) where the information can be further processed and/or transmitted to additional devices/systems.

Also described herein is executable logic for adapting devices to transmit ultrasonically. The executable logic may comprise non-transitory computer-readable storage medium storing a set of instructions capable of being executed by a processor such as a processor of a smartphone, that when executed by the smartphone causes the smartphone to send information ultrasonically (e.g., using a piezo).

This executable logic may also be referred to as an adapter for adapting medical sensing devices so that they may ultrasonically transmit biological parameter information to a telecommunications device for further processing. Also described are systems and/or subsystems for use with a telecommunications device so that the telecommunications device can receive and translate an ultrasonically encoded health metric information signal. These subsystems may include client software (e.g., applications) to be run on the telecommunications device (e.g., phone) to translate the ultrasonic health information (or biological parameter) signal into a digital signal that can be uploaded, stored, and/or analyzed by the telecommunications device.

A medical sensing device may be any device for receiving biological parameters, such as patient vitals. The biological parameters may also be referred to as biometric data. For example, a medical sensing device may be a thermometer, blood pressure transducer, glucose monitor, pulse oximeter, pulse rate meter, pedometer, etc. The medical sensing devices or systems referred to herein are typically digital systems because they may display a numeric (e.g., digital) representation of the biological parameter. For example, the devices may convert analog biological parameters (e.g., temperature, blood sugar, blood pressure or any other health metric information) into digital signals that may be displayed or otherwise presented to the user. For example, a medical sensing system may include a digital thermometer for taking a subject's temperature, a blood cuff for presenting patient blood pressure, a blood sugar (glucose) monitors, a pulse oximeter, or the like, including combinations of these devices. Medical sensing systems or devices for home use are of particular interest, and especially those having sensors that monitor or collect biological parameters from patients and present the information on a display.

As described in greater detail below, in some variations the devices and systems format and/or encode the information so that it includes a hybrid of both digital (e.g., extracted and/or alphanumeric) information and analog (e.g., graphical) information. As used herein the phrase ‘analog’ refers to information that is sequentially ordered and may be graphically displayed to show a change or trend. The analog information may refer to a variable physical level that is quantified (e.g., a variable that varies over time). The actual information may be digital (e.g., by converting from continuous to discrete), but it may still be referred to as “analog” herein because it represents a change in one or more parameters over time, distance, or some other variation.

Any of the information transmitted as an ultrasonic signal (e.g., analog, digital, hybrid digital/analog, etc.) may be encrypted. For example, the information may be encrypted so that they can be decrypted using an encryption key. The encryption key may be displayed or otherwise made available on or by the device transmitting the ultrasonic signal. In general, the encryption key may be input into a telecommunications device so that that particular device is then paired with the device including the ultrasonic modem and may receive and decrypt the information using the encryption key. Encryption of data may allow protection of patient-sensitive information by sound (e.g., ultrasound). Encryption may also reduce the noise in the system, as it may limit the signals received to those that are properly encrypted.

Although the methods and systems described herein may allow an encryption key for decrypting an encrypted signal to be displayed on the device transmitting the encrypted information, the methods may provide security because the encryption key may be encoded in a non-sonic format, including an optical format. For example, the encryption key can be printed or shown on a surface (including visible or, in some variations, covered but exposable, surfaces) of the transmitting device. The encryption key could also be encrypted in a manner that the receiving device (e.g., the mobile communications device configured to read/receive the encryption key) is competent to understand. For example, the encryption key printed on the device may be read by a smartphone; the smartphone may translate the encryption key image into a usable encryption key that can then be used to decode/decrypt the transmitted ultrasound signal. An encryption key may be any piece of information (parameter) that determines the functional output of a cryptographic algorithm or cipher. Without a key, the receiving device attempting to decode the transmitted signal would produce no useful result. A key may specify the particular transformation of signal into ciphersignal, or vice versa during decryption/encryption. Many types of encryption and encryption keys are known to those of skill in the art, and may be used with the methods and apparatuses described herein.

As used herein biological parameters or information may include any patient information that is processed, sensed, and/or calculated by a medical sensing system, and particularly digitally encoded biological parameters. For example, biological parameters may include temperature, blood pressure, blood sugar level, pH, oxygenation, pulse rate, respiratory rate, or any other biological measurement, particularly those relevant to medical case, including diagnosis and health monitoring.

As used herein telecommunications devices includes smartphones (e.g., iPhone™, droid™ or other personal communications devices), tablet computers (e.g., ipad™, tablet PCs, or the like), and/or desktop computers that include (or may be adapted to include) a microphone capable of receiving ultrasonic sound. A telecommunications device may include logic for translating the digital signal encoded by the ultrasonic sound into a digital signal that can be displayed, uploaded/transmitted, stored, and/or analyzed.

Thus, in some variations, described herein are medical sensing devices for ultrasonically transmitting digital biological parameters. In some variations the device may include: a sensor for detecting a biological parameter from a patient; a processor for encoding a digital representation of the biological parameter as an ultrasound sound signal; and an ultrasonic transducer for transmitting an ultrasonic sound signal from the processor.

For example, the sensor may be a transducer for transducing a biological parameter (temperature sensor, pressure sensor, etc.). The device may also include a controller (e.g., microcontroller) for processing signals from the sensor(s). The processor may include a signal generator that generates a signal from sensed and/or processed patient biological parameter information; the signal may be encoded for transmission. The signal may be encoded as a digital packet (e.g., words, bytes, etc.). For example, the signal may include a start bit, stop bit, information bit(s) identifying the type or source of the biological parameter (e.g., packet identifier), a digital representation of the biological parameter and in some variations a cyclic redundancy check (CRC) portion. In some variations, the signal (including the biometric measurement or data portion) can have a time and/or date stamp.

As mentioned, in some variations the system may be configured to encrypt the information and transmit only the encrypted information; the telecommunications device may be configured to receive the encryption key either directly (e.g., by taking and/or analyzing a figure describing the encryption key.

In some variations, the system or devices may be configured so that the measurement is made at time x and stored on the device (e.g., thermometer, glucometer, etc.) and transmitted to the telecommunications device (e.g., smartphone or tablet) ultrasonically at a later time, and eventually uploaded (e.g., to the cloud). In some variations, several time/date stamped measurements may be stored on a device and could be transmitted together in a burst to the telecommunications device. As described in greater detail below, although the device may be primarily one-way (e.g., sending data from the biometric device to the telecommunications device) in some variations the devices may be configured to receive at least a confirmation signal and/or an indicator of the proximity of the telecommunications device. In some variations the ultrasonic transducer may also be configured to receive a confirmation signal from the telecommunications device. Confirmation may indicate that the telecommunications device received a sent message (data) or that the telecommunications device is ready to receive the sent data, or both.

The ultrasonic transducer may be any appropriate transducer, including a piezo crystal transducer.

In some variations, a system for ultrasonically transmitting digital biological parameter includes: a medical sensing device having: a sensor for detecting a biological parameter, a processor for encoding a digital representation of the biological parameter as an ultrasound sound signal, and an ultrasonic transducer for transmitting the ultrasonic sound signal; and client control logic configured to be executed by a telecommunications device and to receive the ultrasonic sound signal and convert it back to a digital representation of the biological parameter.

The processor may convert some or all of the digital biological parameter signal (which is typically a numeric value) into an ultrasonic signal by the use of any appropriate signal processing technique, including, but not limited to, frequency-shift keying.

The client control logic may also be referred to as software (though it may be software, hardware, firmware, or the like), or a client application. The client control logic may execute on a telecommunications device. The client control logic may also include components for passing the digital representation of the biological parameter on to other devices, e.g., uploading it to a website or server, for example. In some variations the client control logic may be configured to display or otherwise present the information locally on the telecommunications device.

Also described herein are systems for transmitting a digital health parameter, the system comprising: an ultrasonic transducer, wherein the ultrasonic transducer is capable of transmitting signals in an open-air environment at frequencies above about 17 KHz (e.g., 19 KHz, or centered around 20 KHz); and a signal generator configured to generate an ultrasonic signal corresponding to a digital representation of a biological parameter, wherein the identifier is associated with at least one frequency above about 17 KHz (e.g., 19 KHz, or centered around 20 KHz).

As an example, described herein are digital thermometer to ultrasonically transmit digital temperature information to a telecommunications device for further processing and transmission. The digital thermometer may include: a temperature sensor for sensing patient temperature; a signal generator for generating a signal corresponding to a digital representation of the patient temperature; and an ultrasonic transducer for transmitting the digital representation of the patient's temperature as an ultrasonic signal comprising one or more frequencies above 19 KHz. The thermometer may include an encryption key on the outside of the thermometer that may be imaged and/or viewed by a user and/or a telecommunications device configured to receive the ultrasonic signal.

In general, described herein are digital ultrasonic modem devices for ultrasonically and securely transmitting digital data. Such devices may include: a microprocessor; an ultrasonic transducer; an encryption key located on the device; and ultrasonic transmission logic that configures digital data for acoustic transmission by the ultrasonic transducer at frequencies at or above 17 KHz, the ultrasonic transmission logic further configured to encrypt the digital data according to the encryption key.

Any appropriate ultrasonic transducer may be used. For example, the ultrasonic transducer may be a piezoelectric speaker.

As mentioned, the encryption key may be visibly marked on the device, and may be configured as an alphanumeric code, a symbol, or the like. For example, the encryption key may be configured as a bar code, a QR code, etc.

Any of the systems described herein may be configured as systems for secure ultrasonic transmission of data, and may include: an ultrasonic communications device comprising an ultrasonic transducer, an encryption key located on the ultrasonic communications device, and ultrasonic transmission logic that configures digital data for acoustic transmission by the ultrasonic transducer at frequencies at or above 17 KHz, the ultrasonic transmission logic further configured to encrypt the digital data according to the encryption key; and decrypting logic executable on a telecommunications device, wherein the telecommunications device comprises a receiver for receiving an ultrasonic signal from the ultrasonic communications device, and wherein the decrypting logic is configured to receive the encryption key and apply the encryption key to decrypt the ultrasonic signal.

In general, the encryption key may be visible on the ultrasonic communications device, packing for the device, or the like.

In any of these variation described herein, the telecommunications device may include an input for inputting the encryption key, which may provide information to the decryption logic. For example, the input may be a camera for taking an image of the encryption key (e.g., bar code, QR code, etc.) and determine the encryption key therefrom. In some variations the input comprises a manual input (e.g., keypad, touchscreen, etc.) for manually entering an encryption key.

Also described herein are methods of securely transferring information using ultrasound. For example, in some variations the method includes receiving an encryption key that is present on an outer surface of an ultrasonic communication device; receiving an encrypted ultrasonic signal from the ultrasonic communications device; and decrypting the ultrasonic signal with the encryption key.

In some variations, the step of receiving an encryption key comprises taking the encryption keys from the outer surface of ultrasonic communications device. Decrypting the ultrasonic signal may include decrypting the ultrasonic signal in a telecommunications device. As mentioned, receiving the encryption key may comprise imaging the encryption key using a camera on the telecommunications device.

In general, any of the systems described herein may use hybrid digital and analog encoding. For example, a device for transmission of both digital and analog ultrasonic data (hybrid digital and analog data) may include: a microprocessor; an ultrasonic transducer; and hybrid transmission logic configured to generate a signal comprising digital data appended to analog data, for acoustic transmission by the ultrasonic transducer at frequencies at or above 17 KHz.

As mentioned above, the information maybe encoded with frequency shift keying (FSK); the FSK digital data may be appended to an analog data that has not been encoded by FSK but has been frequency modulated to form a hybrid digital/analog signal.

In any of these variations, the device may include a sensor for detecting a biological parameter from a patient, and/or a microprocessor configured to extract the digital data from the analog data. In some variations, the digital data comprises calibration data for the analog data (e g , minimum, maximum, variable interval (e.g., time interval), scale, etc.). The analog data may comprise any appropriate signal, typically measured from a device sensor, such as: an EEG, a subject's temperature over time, a subject's glucose level over time, a subject's blood pressure over time, a subject's oxygen level over time, or a subject's physical activity over time, etc.

Also described herein are methods of transmitting a hybrid digital and analog signal using ultrasound. For example, a method may include: generating an ultrasound signal comprising digital data encoded with frequency shift keying (FSK) appended to an analog signal comprising a frequency modulated signal that is modulated at a frequency above 17 KHz; and acoustically transmitting the signal using an ultrasonic transducer.

The method may also include detecting a biological parameter from a patient, wherein the analog signal comprises the biological parameter. The method may also include extracting the digital data from the analog signal. The analog signal may comprise: an EEG, a subject's temperature over time, a subject's glucose level over time, a subject's blood pressure over time, a subject's oxygen level over time, or a subject's physical activity over time.

In some variations, the method also includes the step of receiving the ultrasound signal on a telecommunications device having an ultrasonic audio pickup.

In any of the variations described herein, the ultrasound signal may be stored before transmitting. Any of the variations described herein may be encoded with an error correction code. The method may also include retransmitting the ultrasound signal; the signal may be retransmitted a fixed number of times or it may be retransmitted continuously. In some variations two-way communication may be used between the ultrasonic communications device and the telecommunications device including executable logic for receiving and/or decrypting the ultrasonic signal. Thus, in some variations the telecommunications device may be configured to transmit a signal back to the ultrasonic communications device. The ultrasonic communications device may include a receiver, or it may be adapted to receive a signal on the transmitter (e.g., piezo).

### BRIEF DESCRIPTION OF THE DRAWINGS

FIG. 1 is a pictorial representation of the human range and thresholds of hearing from http://en.labs.wikimedia.org/wiki/Acoustics.

FIG. 2 is a pictorial representation of hearing loss with age from www.neuroreille.com/promenade/english/audiometry/audiometry.htm.

FIG. 3 is an audiogram illustrating the intensity and frequency of common sounds from www.hearinglossky.org/hlasurvival1.html.

FIG. 4A is a schematic representation of a system that is configured to ultrasonically transmit digital data encoding one or more biological parameter to a telecommunications device such as a smartphone.

FIG. 4B is a schematic representation of a system including a medical sensing device that is configured to ultrasonically transmit digital data encoding one or more biological parameter to a telecommunications device such as a smartphone.

FIG. 5 shows one variation of a digital signal that has been encoded using frequency key-shifting in an ultrasound range, as described.

FIG. 6 is an exemplary flowchart illustrating one method of transmitting encoded data as an ultrasound signal.

FIGS. 7A-7E are exemplary flowcharts of a method for transmitting a signal (e.g., packet transmission) as an ultrasound signal.

FIG. 8 shows one example of flowchart of a demodulator and packet decoder for a receiver configured to receive and decode data that is transmitted ultrasonically as discussed herein.

FIG. 9A shows one exemplary format for a hybrid digital and analog ultrasonic data format.

FIG. 9B shows another exemplary format for a hybrid digital and analog ultrasonic data format.

FIG. 10 is a schematic illustration of a system for secure ultrasonic transmission of data including an ultrasonic communications device with an ultrasonic transducer and an encryption key located on the ultrasonic communications device and decrypting logic executable on a telecommunications device, wherein the telecommunications device comprises a receiver for receiving an ultrasonic signal from the ultrasonic communications device.

### DETAILED DESCRIPTION

In general, described herein are apparatuses (e.g., devices and systems) for ultrasonically transmitting information (e.g., biological parameter information) from an ultrasonic transmission device to a telecommunications device that can then process and/or transmit (e.g., broadcast, upload, retransmit, etc.) and/or store the biological parameter information. The ultrasonic transmission device may be any device that includes an ultrasonic modem for encoding and transmitting information as an acoustic ultrasonic signal.

In particular, described herein are apparatuses in which the ultrasonic signal is securely transmitted and may be decrypted using an encryption key that is present on the apparatus. Also described herein are systems, methods and device for easily pairing an ultrasonic transmission device to a telecommunications device using an encryption key. For example, in some variations the telecommunications device may read (e.g., take an image of) an encryption key that is displayed as in image (picture, text, mark, etc.) on the ultrasonic transmission device. This technique may be readily performed by taking an image of the encryption key or a representation containing/encoding the encryption key (e.g., bar code, QR code, etc.) with the receiving device (e.g., a mobile telecommunications device) and determining the encryption key from the image. Executable logic running on the receiving device (e.g. decryption logic) may be configured to interpret and apply this encryption key to decrypt ultrasound signals transmitted by the apparatus.

Also described herein are apparatuses that encode signals, and particularly biological signals, as hybrid ultrasound signals (or signals that may be transmitted ultrasonically) comprising both digital and analog components. These signals may be referred to herein as “hybrid” ultrasound signals, because they have combined digital data (typically data extracted from our about the biological signal) and analog data. For example, an apparatus capable of ultrasonically transmitting biological parameter information may include a sensor for sensing a biological parameter (e.g., vital sign), a processor for configuring a representation of the biological parameter as a “digital” ultrasonic signal, an analog signal, or a hybrid digital/analog signal, and a transducer for transducing the ultrasonic signal so that it can be open-air transmitted to a telecommunications-capable device (e.g., smartphone). The processor may part of, controlled by or in communication with a controller (e.g., a microcontroller). The telecommunications-capable device (telecommunications device) typically includes a receiver (audio receiver) able to receive an audio signal in the ultrasonic range, and a processor for converting the ultrasonic signal back into an electronic signal for further processing or transmission.

It is to be understood that the invention is not limited in its application to the details of construction, experiments, exemplary data, and/or the arrangement of the components set forth in the following description. The invention is capable of other embodiments or of being practiced or carried out in various ways. Also, it is to be understood that the terminology employed herein is for purpose of description and should not be regarded as limiting.

In the following detailed description of embodiments of the disclosure, numerous specific details are set forth in order to provide a more thorough understanding of the disclosure. However, it will be apparent to one of ordinary skill in the art that the concepts within the disclosure can be practiced without these specific details. In other instances, well-known features have not been described in detail to avoid unnecessarily complicating the description.

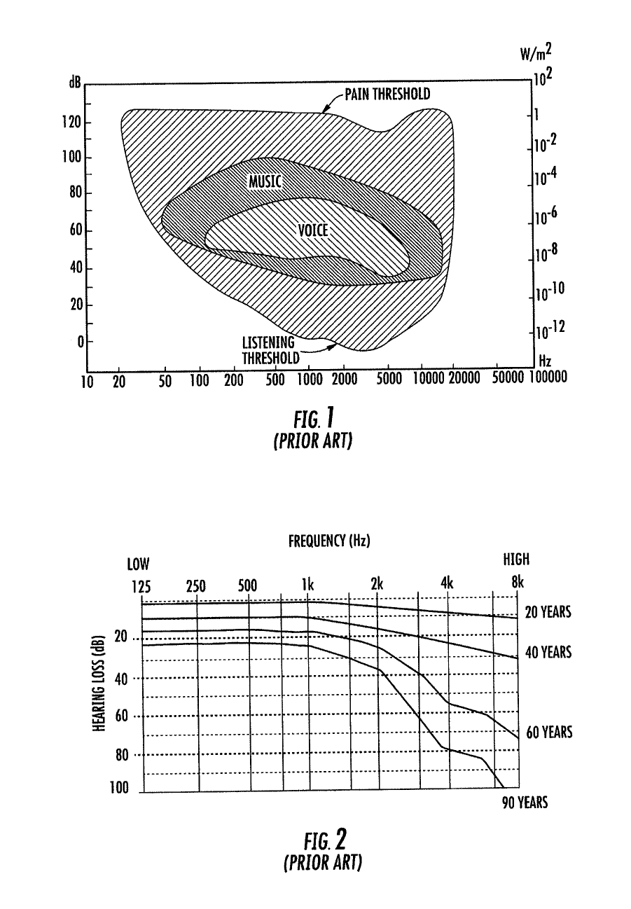

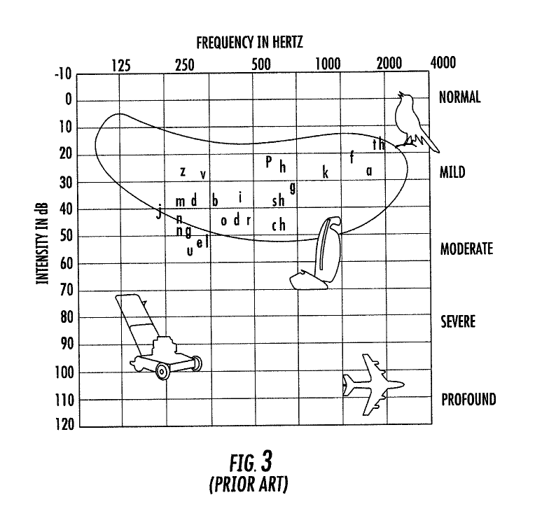

The human hearing range is often referred to as 20 Hz to 20 kHz. A maximum aural range in children, under ideal laboratory conditions, is actually as low as 12 Hz and as high as 20 kHz. However, as shown in FIG. 1, the threshold frequency, i.e. the minimum intensity detectable, rises rapidly to the pain threshold between 10 kHz to 20 kHz. Thus, sounds above about 16 kHz must be fairly intense to be heard. Almost immediately from birth, the threshold sound level for these higher frequencies increases. As shown in FIG. 2, an average 20 year old has lost about 10 dB in the 8 kHz range, while at age 90, the average person has lost over 100 dB at this frequency.

An example product using very high frequency sound is the Mosquito alarm, a controversial device emitting an intentionally annoying 17.4 kHz alarm and used to discourage younger people from loitering. Due to adult hearing loss at this frequency, it is typically heard only by people less than 25 years of age. Similarly, students make use of the adult hearing loss by using “mosquito” ringtones in the 15-17 kHz on their cell phones during school. The students can hear the “mosquito” ringtones while their adult teachers cannot. The term “ultrasonic” typically means above the range perceived by humans. However, as demonstrated, the upper limit of hearing frequency varies with individuals and with age generally. Because of the differences in this upper limit, the term “ultrasonic” as used herein and in the appending claims may refer to “sound frequencies of 17 kHz or greater.” For examples, the sound range may be greater than about 18 kHz, greater than about 19 kHz, between about 17 kHz and about 40 kHz, between about 17 kHz and about 30 kHz, etc.

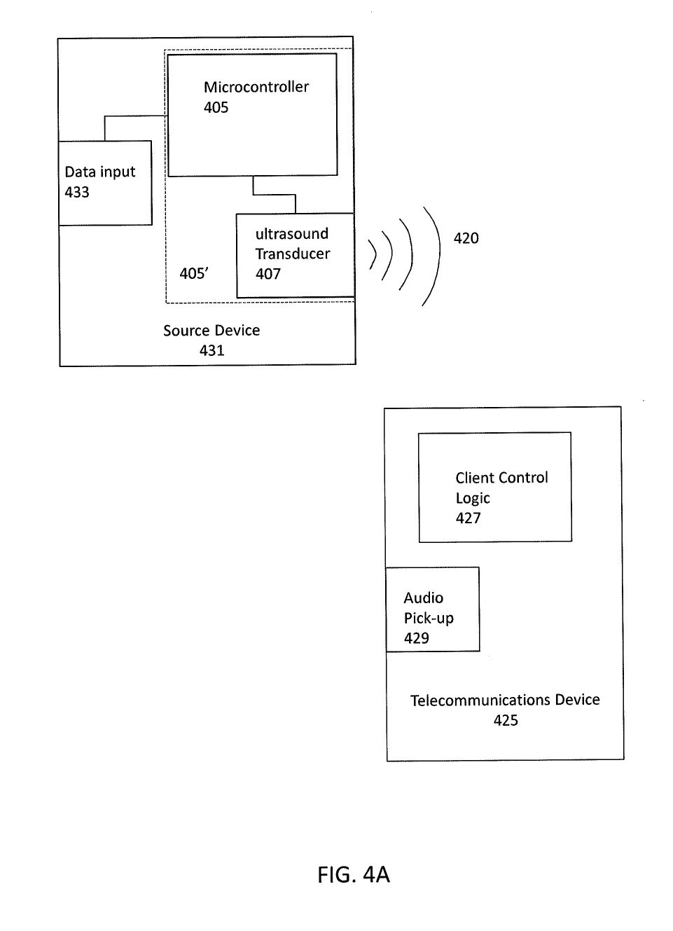

Interestingly, however, there is very little ambient sound or noise above about 10 kHz. Referring to FIG. 3, most everyday sounds occur at frequencies below about 4 kHz. Thus, use of signals in the ultrasonic range is not only silent to those around, but also provides a very desirable signal to noise ratio (SNR).

Acoustic engineers safely assume that any frequency above about 20 kHz will have no effect on the perceived sound and they typically filter everything above this range. Sounds below 20 kHz but still in the ultrasonic range are of little concern, and standard sampling procedures have been established accordingly. It is generally understood that sampling an analog signal, whether a radio signal or audible sound signal, requires a sampling frequency ƒₛ such that ƒₛ/2 >ƒ, wherein ƒ is the sinusoid frequency. For this reason, sound systems are designed to sample the sound at the now standard sample rate of 44.1 kHz, set somewhat higher than the calculated Nyquist-Shannon sampling rate of 40 kHz for a 20 kHz sound upper limit Actual demodulation of an FM narrow band signal in the ultrasonic range, using existing demodulation procedures, computers, telephones, cell phones, stereo sound systems, etc., would result in very poor reproduction of the original signal. This is unfortunate because, as discussed above, a carrier signal in the ultrasonic range would also have a very low signal to noise ratio due to the fact that there is very little natural “noise” at these higher frequencies.

The devices, methods and systems for measuring physiological signals (e.g., biological parameters) and transmitting digital information about those measurements wirelessly and soundlessly use ultrasonic signals having a much improved signal to noise ratio compared to traditional transtelephonic methods. Also provided are methods and algorithms to receive and demodulate the ultrasonic signals with excellent accuracy using existing computer and smart phone technology.

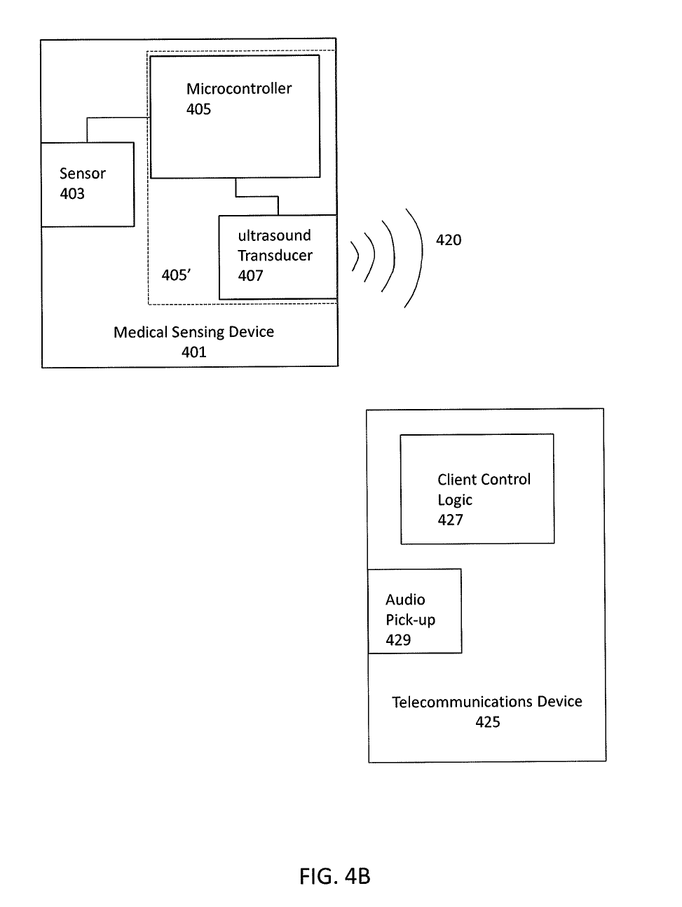

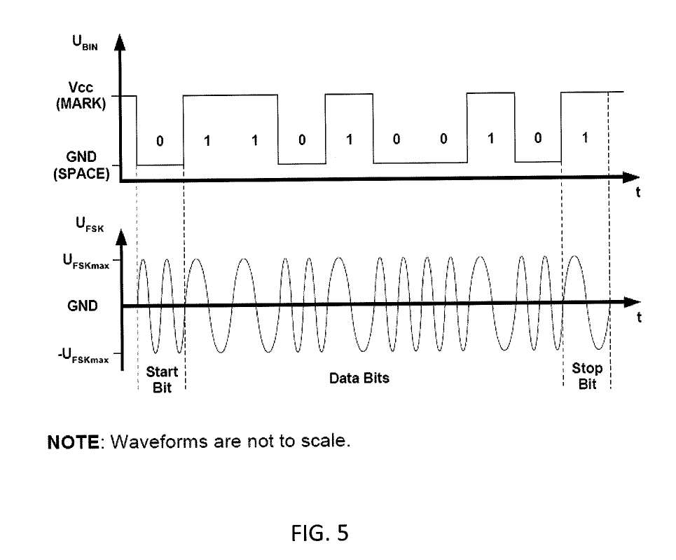

FIG. 4A shows a schematic overview of a system including a data input 433 (e.g., providing any sort of digital information) and a microcontroller/microprocessor 405. The microcontroller may include or be coupled with a processor for encoding a representation of a biological parameter (digitally and/or analog encoding), and this encoded signal may be converted to an ultrasound signal as descried in more detail below. For example, the encoded signal may be transmitted ultrasonically by an ultrasonic transducer 407. In some variations the microprocessor (and/or microcontroller) and the transducer may be coupled together or formed as part of the same component 405′, alternatively, the microcontroller may include a piezo/speaker element. This ultrasonic signal 420 may then be received by a receiving device (e.g., a mobile telecommunications device 425) having an audio pick up (receiver) 429. The telecommunications device 425 may run client control logic 427 preparing the telecommunications device to receive and translate the ultrasonic signal so that it can be processed, e.g., converting it back to an electronic signal, interpreting which type of signal it is (e.g., pulse rate, temperature, etc.), and the like.

FIG. 4B shows a schematic overview of a system including a medical sensing device 401 (e.g., a thermometer, ECG sensor, blood glucose monitor, or the like) that has a sensor 403 for detecting a biological parameter from a patient (e.g., temp, electrocardiogram(s), pulse rate, blood glucose, etc.) and a microcontroller 405. The microcontroller may include or be coupled with a processor (microprocessor) for encoding a digital and/or analog representation of a biological parameter for conversion to an ultrasound signal as descried in more detail below. For example, the encoded signal may be transmitted ultrasonically by an ultrasonic transducer 407. This ultrasonic signal 420 may then be received by a receiving device (e.g., mobile telecommunications device 425) having an audio pick up (receiver) 429. The receiving device 425 may run client control logic 427 preparing the receiving device to receive and translate the ultrasonic signal so that it can be processed, e.g., converting it into an electronic signal, interpreting which type of signal it is (e.g., pulse rate, ECG, temperature, etc.), filtering (or otherwise processing) the signal, analyzing the signal, storing the signal, and/or broadcasting the signal, or the like.

Thus, medical sensing device 401 in this example includes a sensor (or sensor assembly) configured to sense one or more physiological signals, such as temperature, pulse, pressure (e.g., blood pressure), electrocardiogram(s), or the like. Multiple sensors may be used. The sensor(s) may produce electrical signals representing the sensed physiological signals and these signals may be converted to a signal or signals that input to microcontroller or other associated components. This signal may typically be displayed on the device 401 (not shown) and may also be encoded as part of a signal that can then be ultrasonically encoded (e.g., by a technique such as frequency shift keying) to an ultrasonic sound and emitted from the device. The encoding of the signal may be performed by any appropriate circuitry, including, for example a microcontroller such as the MSP430 (e.g., the AFE4110 from Texas Instruments).

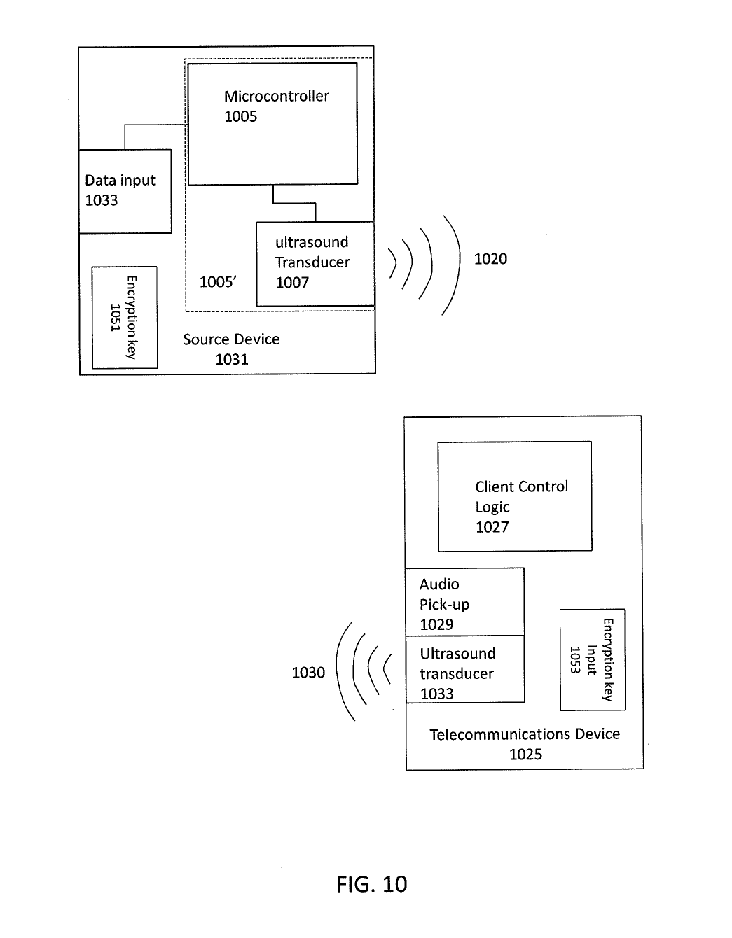

When encoding the signal(s) for transmission, a center frequency or multiple center frequencies may be used. For example, a center frequency may be selected from any appropriate ultrasonic frequency, including (but not limited to) 20 KHz. In some variations the medical sensing devices described herein are configured as transmit only, so that data is transmitted to (but not received from) a telecommunications devices. In some variations, the medical sensing devices are configured to both send and receive ultrasonic (sound) frequency information (see, e.g., FIG. 10). Further, in some variations, multiple channels (frequency channels) may be used.

In one embodiment, the ultrasonic signal has a center frequency in the range of from about 17 kHz to about 40 kHz; about 17 kHz to about 30 kHz; about 17 kHz to about 24 kHz; about 18 kHz to about 30 kHz; about 18 kHz to about 24 kHz, etc. In another embodiment, the frequency modulated ultrasonic signal has a center frequency in the range of from about 20 kHz to about 24 kHz.

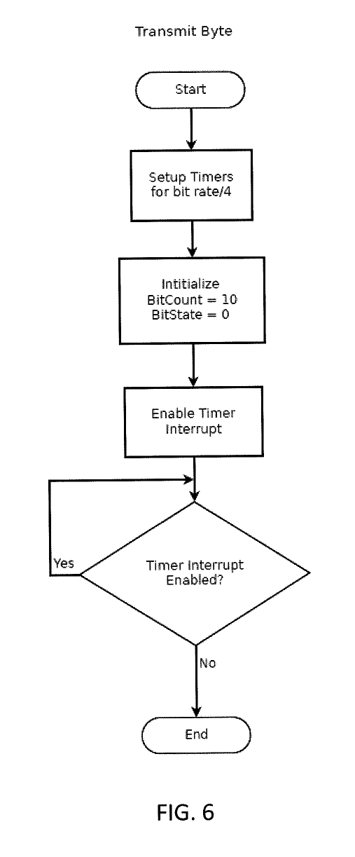

FIG. 5 shows one variation of a digital signal that has been encoded using key-shifting. In this variation the ultrasound signal is modulated at two different frequencies, one indicating high (“1”) and one indicating low (“0”). For example, the frequencies for 0 and for 1 may be selected to be centered around 20 kHz (e.g., 19.5 kHz and 20.5 kHz). This may be referred to as a digital ultrasound signal, in which different frequency values indicate “1” or “0” in a digital signal.

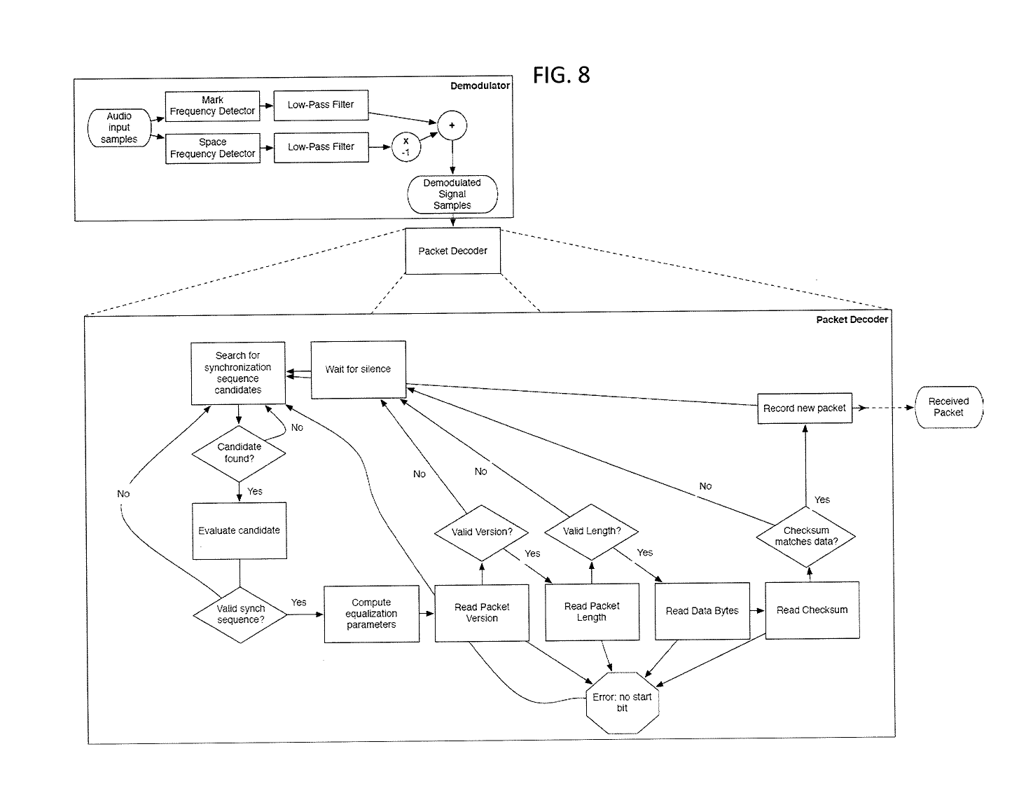

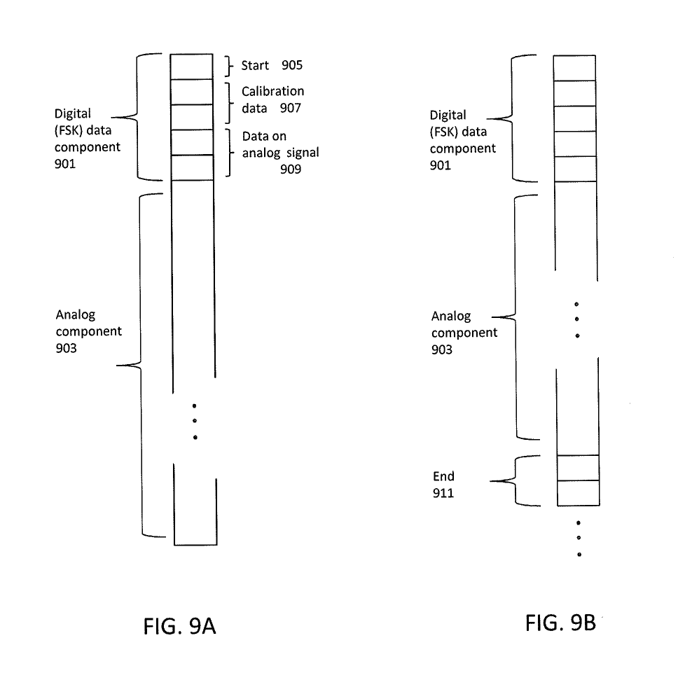

The sensor can include any suitable sensor operative to detect a physiological signal that a user desires to monitor. Nonlimiting examples of such physiological signals include, but are not limited to, respiration, heart beat, bioelectric phenomena (ECG, EEG, etc.) heart rate, pulse oximetry, photoplethysmogram (PPG), temperature, etc. A respiration detector can be used. Heart beat and heart rate can be detected as well. For example, the oxygenation of a person's hemoglobin can be monitored indirectly in a noninvasive manner using a pulse oximetry sensor, rather than measuring directly from a blood sample. The sensor may be placed on a thin part of the person's body, such as a fingertip or earlobe, and a light containing both red and infrared wavelengths is passed from one side to the other. The change in absorbance of each of the two wavelengths may be measured and the difference used to estimate oxygen saturation of a person's blood and changes in blood volume in the skin. A photoplethysmogram (PPG) can then be obtained using the pulse oximeter sensor or with an optical sensor using a single light source. The PPG can be used to measure blood flow and heart rate. A digital representation of this data may be used and passed on as described herein. In some variations (described in reference to FIGS. 9A and 9B, below), analog information may also be encoded and/or appended to digital information to form a hybrid of analog and digital information that is sent by the ultrasonic transmission device.

In some variations a converter assembly converts the electrical (e.g., digital, analog, etc.) encoding of the biological parameter to an ultrasound signal that can be transmitted. In the embodiment shown in FIG. 4, the converter assembly includes an ultrasound transducer 407 for outputting ultrasonic signals. Nonlimiting examples of suitable ultrasonic transmitters (including transducers) include, but are not limited to, miniature speakers, piezoelectric buzzers, and the like.

Within the receiving (e.g., telecommunications device 425), the ultrasonic signals can be received by, for example, a microphone 429 in a device such as a smartphone, personal digital assistant (PDA), tablet personal computer, pocket personal computer, notebook computer, desktop computer, server computer, and the like.

The volume of the signal may be kept low to preserve power, although higher volumes are also possible because the ultrasound is essentially inaudible. For example, the volume of the signal can be further increased at the ultrasonic frequencies, without concern for “listeners” present, because they cannot hear it. Further, the signal may be encoded to prevent other device (not paired with the ultrasonic transmitting device) from receiving and understanding the signal.

As mentioned above, the telecommunications device may include client logic (e.g., software) for receiving and processing the ultrasound signals. Thus, the device may comprise a non-transitory computer-readable storage medium storing a set of instructions capable of being executed by the receiving device. For example, software may configure the smartphone to decode the ultrasound signal. Processing of the data may provide additional information related to the user including the type of the information (e.g., the nature of the biological parameter). For example: the signal may be encoded so that it contains (e.g., after a start identifier): 10 pulses indicating that it is a thermometer reading (e.g., 4 digits coming with last being after the decimal place); 12 pulses indicating it is a blood pressure reading (e.g., 3 digit systolic pressure, 3 digit diastolic pressure and 3 digit pulse rate); 14 pulses indicating that it is pulse oximeter data (e.g., 3 digit O2 sat and 3 digit pulse rate); 16 pulses indicating that it is glucometer data (e.g., 3 digit blood glucose level), etc. There may be a “separator” between the digits and an EOM (end of message) indicator. In practice, the signal may be sent several times so that a comparison may be performed between the received data for validation.

In one variation, the signal may be encoded so that (assuming 8 bit bytes, plus a start and stop bit): some number of AAs, or 55s to allow sync, a byte that denotes a version number, a one byte length of the remainder of the packet, a one byte packet identifier (0x01 for BP, 0x02 for pulse ox, 0x03 for glucose, etc.), data, and an 8-bit CRC.

As mentioned above, the ultrasound signal transmitted/received may be a hybrid ultrasound signal. For example, in some variations the signal may also include a stretch of analog data (e.g., signal over time, signal over distance, etc.) for transmission with digital information, including information that formats or is extracted from (e.g., scales) the analog data. For example, a signal for transmission by ultrasound from an ultrasonic transmission device may include one or more digital portions and one or more analog portions. The digital portion may include information extracted from the analog signal such as the scaling (e.g., max and/or minimum values), duration, average, etc. Analog, digital and analog and digital (hybrid) signals may be encoded, including encryption-encoded and/or may include error correction codes. In some variations, the signal may include error correction codes only related to the digital portion of the signal.

In general, a hybrid signal as described herein is configured so that the digital and analog components are arranged sequentially in time, and may be centered around the same center frequency or frequencies. In some variations an apparatus may be configured to transmit digital signals at the same time as an analog signal (e.g., on different center frequencies); the digital signal may include information about the transmitted analog signal, as mentioned above (including the center frequency of the analog signal, min/max values, scaling/scalor values, etc.).

As mentioned, the signal can have a time and/or date stamp. In some variations the devices or systems may be configured to take multiple measurements and send them to a telecommunications device as a batch or burst. For example, measurements might be made at times t₁, t₂ etc., and stored on the device (e.g., thermometer, glucometer, etc.) and transmitted to the telecommunications device (e.g., smartphone, tablet, etc.) ultrasonically at a later time (tₙ).

The data may be processed by the telecommunications device and/or uploaded to an external server, etc. (e.g., the cloud).

The baud rate of the transmitted ultrasonic data may be selected to allow rapid transmission. For example, if a baud rate of about 300 baud is used, transmission may take less than a second, even for batched signals. In some variations, the baud rate is around 400.

As mentioned, raw signals from the sensors and derived information can be displayed and stored locally on the receiver (e.g., smartphone), as well as being transmitted to a web server over an internet connection. Software on the web server may provide a web browser interface for real-time or retrospective display of the signals and information received from the smartphone, and also includes further analysis and reporting.

Ultrasound signaling as used herein refers generally to the transmission of information, such as the magnitude of a biological parameter along with the origin of the biological parameter measurement, using ultrasonic signals. As mentioned, these ultrasonic signals may be encoded to allow transmission and processing. The encoded signal may then be transduced into the ultrasonic range by any appropriate method. For example, one or more frequencies may be used corresponding to various signal values, e.g. DTMF or DTMF frequency-shifted into ultrasonic frequencies. Another example of transducing the signal is to use amplitude shift keying. Another example is to use frequency shift keying. Another example is to use phase shift keying. In some embodiments, multifrequency signaling such as spread spectrum communications, or a multifrequency carrier signaling, may be used. An example of multifrequency carrier signaling is to designate a predetermined set of frequencies (for example, between 20 KHz and 22 KHz, or between 20 KHz and 24 KHz, or generally between a lower bound between 19 KHz and 20 KHz and an upper bound equal to or slightly below the Nyquist frequency for the sampling rate of an intended receiver) separated by an interval, such as an interval of between 40 Hz and 100 Hz, such as approximately 65 Hz, and for each such frequency, encode a “1” bit as the presence of a carrier signal, such as a sine wave at the frequency, and a “0” bit as the absence of such a signal. A receiver of such a multifrequency signal may then perform Fast Fourier Transforms or related techniques known in the art to identify whether carriers are available at each relevant frequency, and deduce a set of bits, encoding a number, thereby. In some embodiments of multifrequency carrier signaling, for example when a signal is insufficiently unambiguous, multiple samples may be taken over time and averaged, then the average signal may be processed as described above. In some embodiments of multifrequency carrier signaling, a Viterbi decoder may be used to decode the bit patterns, for example if the frequencies are sufficiently close as to cause interference. In general, techniques known to those skilled in the communications arts, especially with respect to modulation and demodulation (e.g. modems), may be employed. Examples of such techniques include the various modem standards designated as V.x (where x is an integer) promulgated by the International Telecommunications Union, Sector T, which are incorporated herein in their entirety by reference for all purposes.

In some embodiments, a server may perform signal analysis to determine the encoded data, rather than (or in addition) to on the telecommunications device. In some embodiments, signals may be stored at the server and provided to personnel for refinement of transmission and/or reception techniques.

As mentioned above, signaling may be performed by a transmitter. A transmitter may include a hardware system that incorporates a signal generator such as processor, such as a microprocessor, microcontroller, or digital signal processor connected to a memory (for example, DRAM or SRAM, which in some embodiments may be integrated with the processor) containing program instructions executable by the processor, and/or data used by the program. A transmitter may also incorporate persistent memory, such as a flash memory, coupled to the processor and/or incorporated into the processor. The signal generator may generate the ultrasonic signal that is transmitted as described above. In some embodiments, a waveform for transmission may be stored in persistent memory. In some embodiments, a transmitter includes a power supply and/or a battery, or uses the power supply used to power other components on the medical sensing device. As mentioned, the transmitter may include a transducer, for example a piezoelectric transducer that converts electrical impulses to ultrasonic vibrations. A transmitter may include an amplifier coupled (directly or indirectly, for example via an audio Digital-to-Analog Converter (DAC), which in some embodiments may be integrated with the processor) to the processor, which provides electrical impulses through its output to the transducer. In some embodiments, transmitter may include a real-time clock and/or a receiver for receiving broadcast time signals. In some embodiments, transmitter may include an encryptor, which for example may be program instructions executing on processor, or may be separate integrated circuitry. In some embodiments, transmitter may include an error correcting code generator and/or an error detecting code generator, which for example may be software instructions executing on processor, or may be separate integrated circuitry. The techniques described herein regarding transmission and reception of sonic signaling may be performed at a transmitter as described herein in a manner that will be readily understood by those skilled in the art.

In some variations, the transmission from the medical sensing device to the telecommunications device is one-way, which may provide a simplicity of the design, lower expense, lower power consumption, and the like. These advantages are particularly helpful when compared to systems in which the medical sensing device includes an additional receiver (including a microphone for receiving sonic signals, or an antenna). However, in some configurations the apparatus (e.g., medical sensing device) may be adapted for two-way (duplex, half-duplex, full-duplex) communication, e.g., to receive an indicator signal from the telecommunications device without requiring the addition of a receiver such as an antenna or microphone. For example, in some variations a return acknowledgement (ACK) could be implemented using the ultrasonic transducer (e.g., piezo speaker) as a sensor as well as a transmitter (e.g., a 20 kHz sensor). For example, the receiver device (e.g., a mobile telecommunications device such as a smartphone) could produce a short 20 kHz burst after receiving, decoding, and verifying the CRC to signal to the sensor that it received it correctly, indicating that re-transmission is not necessary. In other variations a signal from the receiver device may indicate that it is ready to receive transmission from the transmitting (biometric) apparatus. Pairs or multiples of timed signals/acknowledgements may also be used.

In one example, the devices or systems are configured so that the data that is ultrasonically transmitted includes forward error correction (FEC), allowing the receiver to correct N number of bit errors. This may be particularly useful if the system is configured so that the biometric device (the medical sensing device) is transmit-one (e.g., one-way). FEC may help ensure that the data is received correctly.

As mentioned, data sent by ultrasonic signaling may be processed to include an error correcting code, such as a BCH code, a Constant-weight code, a Convolutional code, a Group code, a Golay code such as a Binary Golay code, a Goppa code, a Hadamard code, a Hagelbarger code, a Hamming code, a Latin Square based code, a Lexicographic code, a sparse graph code such as a Low-Density Parity-Check code, an LT or “Fountain” code, an Online code, a Raptor code, a Reed-Solomon code, a Reed-Muller code, a Repeat-accumulate code, a Repetition code such as Triple modular redundancy code, a Tornado code, a Turbo code, or other error correcting codes known to those skilled in the art. In various embodiments, such codes may be applied in a single dimension or in multiple dimensions, may be combined, and may be combined with error detecting codes such as parity and cyclic redundancy checks. Error correcting codes may be decoded and applied to correct transmission and/or reception errors at a receiver, or at a server receiving communications from a receiver, according to their respective techniques.

### EXAMPLE 1

Digital Thermometer

In one example, a digital thermometer may be configured to include a digital ultrasonic modem. In this example, a digital thermometer based on a Texas Instrument MSP430 digital thermometer has been adapted to include firmware so that it may ultrasonically transmit the temperature reading (digital data) to a mobile telecommunications device (e.g., iPhone). Although this example is specific to the APE 4110 microprocessor (one variation of the MSP 430 microprocessor from Texas Instruments) other microprocessors may be used and similarly adapted with firmware, software and/or hardware to function.

In general, the device may take data (e.g., thermometer temperature readings) and encode them for ultrasonic transmission. The encoded signal may include error checking (e.g., CRC encoding, Hamming codes, etc.) and may be encrypted. For example, the data may be data encrypted using, for example Advanced Encryption Standard (AES). U.S. Pat. Nos. 5,481,255 and 5,452,356 both describe data encryption methods and techniques that may be used with the data described herein.

For example, data received from the thermometer may be encoded and/or encrypted into one or more data packets for transmission. The microprocessor may encode the data and may then transmit the packets by driving the piezo speaker. As mentioned above Frequency Shift Keying (FSK) may be used, in which two separate ultrasonic frequencies (e.g., 18817 Hz and 19672 Hz) are used to transmit Boolean 0 and 1, respectively. The control logic (data ultrasound modem logic) may both configure, encode and encrypt the data and may also control driving the transmission of the prepared packets of encoded/encrypted data by the speaker (e.g., piezoelectric transducer). The control logic may also control the timing of the delivery, so that there is adequate spacing between each data bit. In addition, the control logic may also repeat the transmission and time the start of the transmission.

For example, in one variation the thermometer typically measures temperature, and once the temperature has settled to a value, the thermometer emits an audible beep to alert the user that the value can be read. This thermometer (in the initially unmodified configuration) includes a microcontroller (e.g., the AFE 4110) and a piezoelectric speaker; the microcontroller drives the speaker to emit the beep. By modifying/configuring the microcontroller as described herein to include the control logic for the digital ultrasound modem, the thermometer may be adapted to “wirelessly” (via ultrasound) transmit the thermometer data to a device configured to receive and decode/decrypt the signal such as a smartphone running digital ultrasound modem receiver logic.

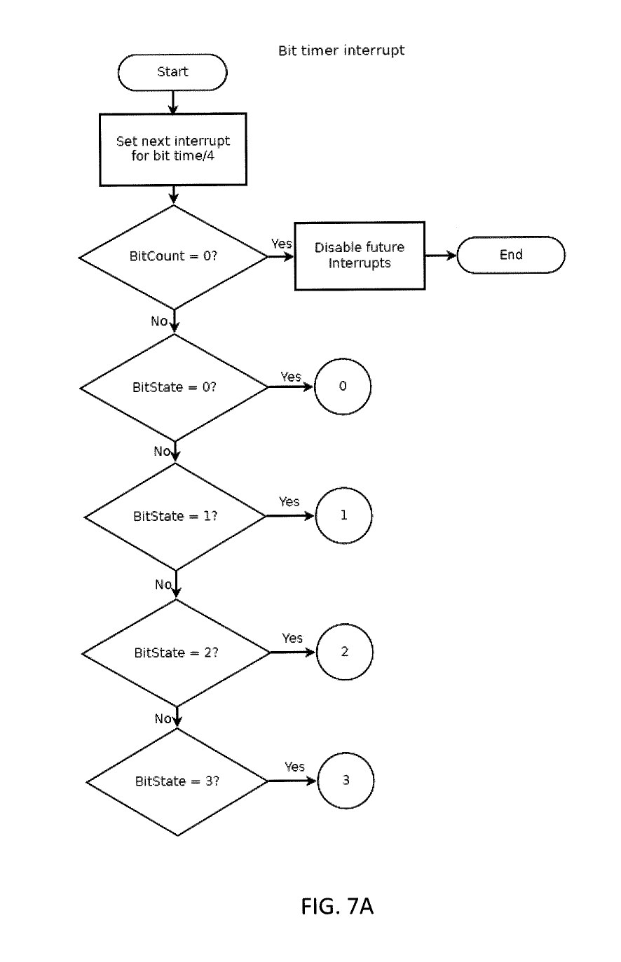

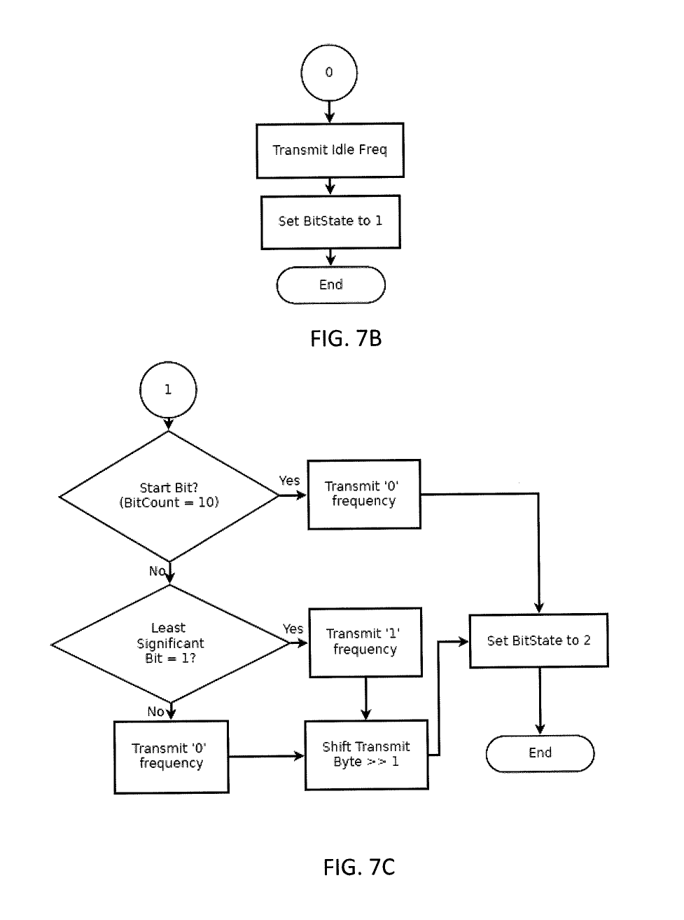

In this example, the microprocessor may include the following (exemplary) code to enable the functionality described above. FIGS. 6 and 7A-7E show flowcharts describing methods for transmitting data.

In any of the systems, device, or methods described herein data (including digital, analog, and/or hybrid digital/analog data) may be compressed before it is encrypted. Any appropriate data compression technique may be used. For example, data compression may be performed using lossy and/or lossless techniques. Known types of lossy and lossless data compression may be used. For example, Lempel-Ziv (LZ) compression and other statistical redundancy techniques may be used for lossless compression. Similarly, lossy data compression techniques may also be applied. The receiver executing the control logic may decompress the data.

Ultrasound Digital Modem Receiver

As mentioned above, a receiver (a digital ultrasound modem receiver) may be used to receive the transmitted ultrasound signal. The receiver may be a dedicated device include a microphone competent to receive ultrasound signals and a processor capable of analyzing the signal (e.g., microprocessor) or it may be a device having microprocessor and microphone that is adapted to receive the ultrasound signal when executing control logic (e.g., digital ultrasound modem receiver logic). In particular, the receiver may be a mobile telecommunications device, such as a smartphone.

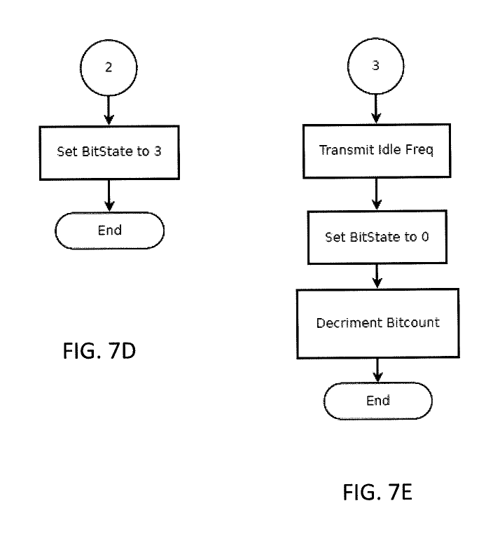

For example, FIG. 8 illustrates one variation of a flow diagram illustrating a method for receiving, demodulating and detecting an ultrasound signal (including digital and/or hybrid ultrasound signals). In this example, the application (the receiving control logic) receives binary-FSK encoded data via a microphone input. For example, the input may be from the microphone on a smartphone. As discussed above, Binary FSK encoding uses two frequencies, a “mark” frequency Fₘ to represent a binary 1, and a “space” frequency Fₛ to represent a binary 0. In this implementation, no carrier is used. The system may also be configured to recognize analog components of the signal. For example, the digital portion of a hybrid signal may indicate when, and for what duration, an analog portion of the hybrid signal, will follow.

The exemplary application consists of two largely independent components: the demodulator, which extracts the mark and space frequency components from the raw audio data, and the packet decoder, which monitors the demodulated signal for packet transmissions and decodes them. These are illustrated in FIG. 8. The demodulator receives audio samples from the microphone hardware at a sample rate S, such that S >2* max(Fₘ,Fₛ). The audio samples are processed by two frequency detectors that calculate the intensity of the mark and space frequency components (respectively) of the received signal. A Goertzel algorithm is used for frequency detection in this implementation. In order to achieve sufficient frequency resolution between the mark and space frequencies, a Goertzel algorithm was applied to a sliding window of G samples, where G=S/abs(Fₘ-Fₛ).

The output of the Goertzel algorithm for the mark and space frequencies is passed to independent low-pass filters, with a passband equal to the baud rate. The filtered output of the space frequency signal is then subtracted from the filtered output of the mark frequency signal. This produces a waveform that is approximately 0 when there is no transmission occurring, rises to a positive value when the “mark” frequency is active, and falls to a negative value when the “space” frequency is active.

This demodulated waveform is then passed to the packet decoder. For each raw audio sample received from the microphone hardware, the demodulator produces a single demodulated sample of the demodulated waveform. The packet decoder receives demodulated samples from the demodulator. The decoder maintains a buffer of the last N samples received, where N is equal to the length of the synchronization sequence. With each new sample, the decoder evaluates the past N samples in the buffer to determine if they contain the synchronization sequence. A two-stage test is used—first a computationally simple evaluation that eliminates most false positives due to random noise, and then a more computationally expensive evaluation that eliminates the rest.

Once a valid synchronization sequence is received, the decoder stores properties of the received signal (e.g. maximum mark/space amplitudes, etc.). These equalization parameters are used to calibrate the decoder thresholds used to read the remainder of the packet. The decoder in this example then reads each encoded byte in turn. It uses the stored equalization parameters to determine a minimum amplitude threshold for the start bit of each byte. Once a valid start bit is received for a given byte, subsequent bits are evaluated based on the sign of the demodulated waveform, with no minimum threshold for decoding.

If no valid start bit is received, the decoder aborts reading the packet and waits for silence, or until a fixed amount of time has passed, before resuming listening for new packets. Each logical byte in the packet is actually transmitted as two encoded bytes—the first containing the Hamming-encoded low nibble of the logical byte, and the second the Hamming-encoded high nibble.

The first logical byte read is the packet version, which is checked against supported version numbers. Next the packet length is read, specifying the number of data bytes to follow. If the packet length exceeds the maximum length for the specified packet version, the packet is rejected. Subsequently, each logical data byte is read.

After the data bytes are read, two logical checksum bytes are read, and the checksum value received is compared to the value computed for the data bytes received. If these two checksum values match, the packet is considered valid, and is made available to the remainder of the application. If they do not match, the packet is rejected. The two logical checksum bytes represent the end of the packet. After receiving the packet, the decoder resumes listening for new packets.

Once data is received (and in some variations decrypted), it may be processed further and/or stored, and/or displayed, and/or transmitted on using any of the communications capabilities of the telecommunications device. For example, the data may be displayed on the smartphone and/or uploaded into a medical database for storage and/or later review.

Although the example above describes a system configured to transmit digital information, the techniques, device and systems described herein may be configured to transmit analog signals as well, and/or analog and digital hybrid signals. In general, the techniques described include the use of a timer (e.g., in the microcontroller) transmitting to a piezo to generate the ultrasound signal. Alternatively, in some variations the system uses a D/A converter to drive a speaker for non-digital output. Further, in some variations the system the output is not a piezoelectric element but is a more traditional speaker (albeit in the ultrasound range). Additional digital to analog (D/A) conversions may take place during transmission.

For example, FIGS. 9A and 9B illustrate one variation of a hybrid digital/analog format that may be used with an ultrasound transmitter. In general, the signal may include a digital component that is modulated or configured for ultrasound modem transmission. For example, the digital signal may be encoded as an FSK signal, and data (e.g., analog data such as biometric data like ECG, blood oxygen/pulse ox, etc.) may be encoded as frequency modulated waveforms that are appended to the digital information.

In some variation the ultrasonic transmission device is configured as a pulse-ox measuring/monitoring device. In this example, information taken from the pulse-ox may be examined to extract information, such as the minimum, maximum, analog signal duration, etc. and may be digitally encoded an placed (using one or more encryption and/or error correction codes) in a buffer and/or transmitted by ultrasound. The analog signal may be combined with the digital signal (or extracted signal) that can be sent to the transmission element and received by a telecommunications device. In the example of a device configured as a pulse oximetry device (e.g., plethsmograph), the pulse oximetry device prepares the hybrid data/analog signal by determining from the analog signal (e.g., a time varying pulse oximetry signal) the peak, minimum, duration, time interval, etc. of the analog signal. Thus, the hybrid signal may include the extracted or tagging digital information as well as a waveform (or waveforms) taken from the device.

The signals may be sent encrypted by a device or user specific identification code. In general any of the devices described herein may encode the data, and an encryption key may be provided so that it can be read and understood by a receiving telecommunications (e.g., phone, tablet, pad, etc.).

There are many potential benefits to transmitting a hybrid analog/digital signal that can be read and understood by the telecommunications device. For example, if a hybrid signal includes a series of values (e.g., min/max) and waveform (e.g., ECG, hear rate, etc.). This kind of hybrid digital/analog system may allow more efficient communication than just FSK value data alone.

For example, variations of ultrasonic transmission devices may include a pedometer, an activity monitor, a heart-rate monitor, etc. In some variations the signal is formatted so that there are a finite number of points in the analog portion. The ultrasound transmitting device may then send a series of data points (including any including calibration points). In one example a graph of heart rate may include 1000 points in 2 seconds (transmission time) representing a graph of biometric data over time. The signal may include digital values (encoded as FSK, for example) and analog (e.g., graphic) data. Such a hybrid signal may include the best characteristics of both digital-only and analog-only signals.

In one example, previously mentioned above, an ultrasonic transmission device is a thermometer that includes the ultrasonic modem elements described above. The ultrasound thermometer device may be configured to include a temperature range of about 95° F. and 106.7° C. for an actual use range. Thus, temperature may be normally transmitted as having 0.1 resolution (e.g., 120 values, so 8 bits may be all that are needed). In devices configured to encode the biometric data in a hybrid signal, the digital component of the signal may be appended first and may include information about the analog signal that follows the digital-only, while the analog signal may be appended or embedded in the rest of the signal and the digital information may be extracted from the digital signal to be included with it. Examples of hybrid signals may include a thermometer device as mentioned above, which displays temperature as a function of time, and measures and/or records and transmits the maximum/minimum temperature, the time measured, etc., finally the signal may also include a temperature waveform showing time course. Other devices and/or signals (hybrid signals) may be include glucose monitor signals (e.g., configuring the ultrasonic transmission device as a glucose meter, etc.), which may send blood sugar signals (digital signals including max, min, etc.) and one or more graphs showing waveforms of blood glucose over time, etc.

Preparing and transmitting a signal to include both analog and digital information may also allow the system to send more data in compressed form as a waveform, which can be very efficient. For example, prototype ultrasonic transmission devices apply a specific sampling rate (e.g., 300 or 500 samples/sec., where each value is a 16 bit binary value). More data can be efficiently sent in compressed form as a waveform. Including extracted information (such as min and max values of the analog signal) in the digital portion of the signal may provide the axis calibration for the analog portion of the signal, e.g., for display.

FIG. 9A shows one variation of a hybrid digital/analog format that may be used as described herein. In this example, the signal includes an initial digital component 901 that is encoded for ultrasound transmission using a technique such as FSK (or any of the other techniques known in the art). The digital information may be broken into bits, byte, words, etc. as appropriate. The size and position of digital information may be predetermined. Error correction codes (e.g., hamming codes, etc.) may be included. In FIG. 9A, the signal includes a start bit or bytes 905, a sequence of calibration data 907 extracted from the analog signal (e.g., max/min), additional data 909 on the analog signal (e.g., type, timing, data stamp/time stamp, etc.). Any other digital information may be included. Thereafter, the signal may include an analog component 903. In FIG. 9A, the analog signal is somewhat open-ended, and may continue for a fixed or unfixed duration; in some variations the entire signal may be repeated for receipt by the telecommunications device. FIG. 9B shows a similar variation of a hybrid signal format, in which the digital component 901 is appended to an analog component 903, and an additional digital component 911 (“end” signal) may be appended at the end. In some variations multiple analog components maybe combined with multiple analog components. As described below, the entire signal may be encrypted prior to transmission.

In some variations hybrid digital/analog formats may be used to encode stored data that has been held by the device (the ultrasonic transmission device) for some amount of time. For example, stored data such as an hours, days, or weeks' worth of data (e.g., biometric data such as pedometer data) may be prepared as an analog signal (graph overt time) that is described/calibrated by the digital data component, and sent to a telecommunications device.

In any of the devices, systems and methods described herein, the ultrasonic signal transmitted by the device may be encrypted. Any appropriate encryption method may be used, including encryption methods that use keys, such as data encryption standard (DES), advanced encryption standard (AES), and the like.

In general, the encryption key specific for a particular apparatus (e.g., ultrasonic transmission device) may be presented on the apparatus (or on the associated packaging, housing, etc. for the device) so that it can be easily accessed by a user of a receiving device (e.g., smartphone). The encryption key may be prepared as a bar code or other machine readable format (e.g., QR code), and particularly readable formats that can be read using the receiving telecommunications device in a different modality than the ultrasound transmission. As used herein, reference to presenting or displaying an encryption key on the ultrasonic transmission device is intended to encompass displaying a prepared representation (and particularly a machine-readable representation) on the ultrasonic transmission device, it's packing or associated structures (e.g., housing, etc.). Presenting typically means presenting in some other medium other than ultrasound, and is not limited to visible presentation. In some variations the encryption key is prepared as a bar code or QR code and printed on the outside of the ultrasonic transmission device so that it can be photographed or scanned by the telecommunications device. The machine executable logic (e.g., client logic, software, firmware, etc.) on the telecommunications device may then determine the encryption key and apply it to decrypt the ultrasonic signal received from the ultrasound communications device.

In this manner, an ultrasonic transmission device may be paired uniquely with a private encryption key that can be read only by a telecommunication device possessing and applying the encryption key. The encryption key (encryption key) may be readily displayed an easily determined by the telecommunications device. Thus, in some variations, each ultrasonic transmission device may have a unique ID that is printed on the device, providing a code that must match with the telecommunications device. Scanning the printed encryption key allows the telecommunications device to decrypt the data.

FIG. 10 illustrates schematically one variation of a system including an ultrasonic transmission device (“source device” 1031) with an encryption key 1051 visible on the body of the device that can be read and applied by the telecommunications device 1025 to decrypt the transmitted ultrasonic transmission. FIG. 10 also illustrates one variation of an apparatus (e.g., device and/or system) in which the ultrasonic transmission device (“source device” 1031) is in two-way (or limited two-way) communication with the telecommunications device. The same principles described herein, including the encryption/decryption, apply to systems/devices configured for on-way (non-duplex) communication of ultrasound signals, as discussed above.

As mentioned above, it may be useful to have communication between the telecommunications device (e.g., smartphone or computer) and ultrasonic transmission devices such as healthcare/fitness sensing devices, home automation and security devices (door and window sensors, remote light switches, etc.), plant water level detectors, etc. For instance, it would be helpful to implement a half-duplex protocol so that the telecommunications device (e.g., smartphone/computer) could provide acknowledgement (ACK) to the sensing device (source device or ultrasonic transmission device) that the data has been successfully received (with correct CRC) and to stop re-transmitting that data. Another use of this half-duplex protocol would be to configure the remote device by sending parameters or information such as calibration data, personal information, etc. from the telecommunications device.

For simple acknowledgement, the piezo/speaker used by the device to transmit data (ultrasonic transmission device) could be used as a frequency tuned sensor. In general a piezo for transmission of sound may also be configured as a receiver. Using a piezoelectric element as a receiving sensor requires a relatively “loud” signal (even if it's inaudible) and thus the signal should be at the resonant frequency of the piezo at which it is most sensitive. The duration or encoding of such a “frequency burst” could be configured so as to be recognized easily by the low power electronics of the healthcare/fitness sensing device. For example, an acknowledgement pulse could be filtered and detected as just a presence of a certain ultrasonic frequency for a predetermined duration.

In some variations, symmetric two-way communication can be accomplished using well-established telephony modem techniques, only changing the carrier frequency into the ultrasonic range. For instance, telephony modem modulation techniques, based on FSK (Frequency shift keying), QAM (Quadrature amplitude modulation), and PSK (Frequency shift keying). These telephony modem techniques assume only two devices are attempting to communicate. Radio frequency protocols can be used to augment the modem protocols to allow for multiple devices to communicate simultaneously without error.

Implementations of such two way communication techniques may include additional processing power in the device sufficient to perform the signal processing necessary to demodulate and decode the received audio. This processing power may require additional battery power as well as physical space in the device. A partial list of existing modem communication standards that could be adapted to ultrasonic communications may include: ITU V.21 (300 bps, FSK), and ITU V.22 (1200 bps, PSK (Phase shift keying)). See, e.g., reference webpages such as: ftp://kermit.columbia.edu/kermit/cu/protocol.html, http://www.lsu.edu/OCS/its/unix/tutorial/ModemTutorial/ModemTutorial.html, http://www.dtic.mil/cgi-bin/GetTRDoc?AD=ADA499556, http://alumni.media.mitedu/˜wiz/ultracom.html, http://nesl.ee.ucla.edu/fw/torres/home/Dropbox/good_paper_mico_controller.pdf, http://edocs.nps.edu/npspubs/scholarly/theses/2010/Sep/10Sep_Jenkins.pdf.

With respect to FIG. 10, the source device may include an additional transducer/microphone for receiving ultrasound signals from the telecommunications device, as well as supporting processing (e.g., microprocessor/microcontroller logic) to control it, interpret communications (which may encoded and/or encrypted) and execute any command functions. Similarly, the telecommunications device may include a speaker (piezo) configured to emit ultrasonic signals.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A digital ultrasonic modem device for ultrasonically and securely transmitting digital data, the device comprising:
a microprocessor;
an ultrasonic transducer;
an encryption key located on the device; and
ultrasonic transmission logic that configures digital data for acoustic transmission by the ultrasonic transducer at frequencies at or above 17 KHz, the ultrasonic transmission logic further configured to encrypt the digital data so that it may be decrypted according to the encryption key.

2. The device of claim 1, wherein the ultrasonic transducer is a piezoelectric speaker.

3. The device of claim 1, wherein the encryption key is visible on the device.

4. The device of claim 1, wherein the encryption key comprises a barcode.

5. The device of claim 1, wherein the encryption key comprises a QR code.

6. A system for secure ultrasonic transmission of data, the system comprising:
an ultrasonic communications device comprising an ultrasonic transducer, an encryption key located on the ultrasonic communications device, and ultrasonic transmission logic that configures digital data for acoustic transmission by the ultrasonic transducer at frequencies at or above 17 KHz, the ultrasonic transmission logic further configured to encrypt the digital data so that it may be decrypted according to the encryption key; and
decrypting logic executable on a telecommunications device, wherein the telecommunications device comprises a receiver for receiving an ultrasonic signal from the ultrasonic communications device, and wherein the decrypting logic is configured to receive the encryption key and apply the encryption key to decrypt the ultrasonic signal.

7. The system of claim 6, wherein the encryption key is visible on the ultrasonic communications device.

8. The system of claim 6, wherein the encryption key is a QR code.

9. The system of claim 6, wherein the telecommunications device comprises an input for inputting the encryption key.

10. The system of claim 9, wherein the input comprises a camera.

11. The system of claim 9, wherein the input comprises a manual input.

12. A method of securely transferring information using ultrasound, the method comprising:
receiving an encryption key that is present on a surface of an ultrasonic communication device;
receiving an encrypted ultrasonic signal from the ultrasonic communications device; and
decrypting the ultrasonic signal with the encryption key.

13. The method of claim 12, wherein receiving an encryption key comprises taking the encryption key from the surface of ultrasonic communications device.

14. The method of claim 12, wherein decrypting the ultrasonic signal comprises decrypting the ultrasonic signal using a mobile telecommunications device.

15. The method of claim 12, wherein receiving the encryption key comprises imaging the encryption key using a camera on a mobile telecommunications device.

16. A device for transmission of both digital and analog ultrasonic data, the device comprising:
a microprocessor;
an ultrasonic transducer; and
hybrid transmission logic configured to generate a signal comprising digital data appended to analog data, for acoustic transmission by the ultrasonic transducer at frequencies at or above 17 KHz.

17. The device of claim 16, wherein the hybrid transmission logic is configured to encode the digital data with frequency shift keying (FSK) and append the FSK digital data to the analog data that has not been encoded by FSK but has been frequency modulated.

18. The device of claim 16, further comprising a sensor for detecting a biological parameter from a patient.

19. The device of claim 16, wherein the microprocessor is configured to extract the digital data from the analog data.

20. The device of claim 16, wherein the digital data comprises calibration data for the analog data.

21. The device of claim 16, wherein the analog data comprises: an EEG, a subject's temperature over time, a subject's glucose level over time, a subject's blood pressure over time, a subject's oxygen level over time, or a subject's physical activity over time.

22. A method of transmitting a hybrid digital and analog signal using ultrasound, the method comprising:
generating an ultrasound signal comprising digital data encoded with frequency shift keying (FSK) appended to an analog signal comprising a frequency modulated signal that is modulated at a frequency above 17 KHz; and
acoustically transmitting the signal using an ultrasonic transducer.

23. The method of claim 22, further comprising detecting a biological parameter from a patient, wherein the analog signal comprises the biological parameter.

24. The method of claim 22, further comprising extracting the digital data from the analog signal.

25. The method of claim 22, wherein the analog signal comprises: an EEG, a subject's temperature over time, a subject's glucose level over time, a subject's blood pressure over time, a subject's oxygen level over time, or a subject's physical activity over time.

26. The method of claim 22, further comprising storing the ultrasound signal before transmitting.

27. The method of claim 22, further comprising encoding the digital data with an error correction code.

28. The method of claim 22, further comprising retransmitting the ultrasound signal.

29. The method of claim 22, further comprising receiving the ultrasound signal on a telecommunications device having an ultrasonic audio pickup.
